SEQUENCE LISTING (13 sequences)

SEQ ID NO: 1 - DNA, 61 bp, Mus musculus
uguuggauggagaacaacucuaggcagaggucucaaaggcuggggcuguguuggacagcaa

SEQ ID NO: 2 - DNA, 39 bp, Artificial Sequence
  AON ADAR102-1; ADAR102-13
cacagccccagccuuugagaccucuguccagaguuguuc

SEQ ID NO: 3 - DNA, 36 bp, Artificial Sequence
  IDUA125; IDUA163; IDUA170; IDUA176; IDUA182; IDUA247; IDUA250; IDUA253; IDUA103; IDUA268
gccccagccuuugagaccucuguccagaguuguucu

SEQ ID NO: 4 - DNA, 18 bp, Artificial Sequence
  primer
ctcacagtcatggggctc

SEQ ID NO: 5 - DNA, 22 bp, Artificial Sequence
  primer
cactgtatgattgctgtccaac

SEQ ID NO: 6 - DNA, 25 bp, Artificial Sequence
  Wild type probe (FAM NFQ labeled)
agaacaactctgggcagaggtctca

SEQ ID NO: 7 - DNA, 25 bp, Artificial Sequence
  Mutant probe (HEX NFQ labeled)
agaacaactctaggcagaggtctca

SEQ ID NO: 8 - DNA, 21 bp, Artificial Sequence
  target RNA-specific primer
ggaaacgtaggttggggtgtg

SEQ ID NO: 9 - DNA, 23 bp, Artificial Sequence
  Pyroseq Fwd2 IDUA primer
agtactcacagtcatggggctca

SEQ ID NO: 10 - DNA, 22 bp, Artificial Sequence
  Pyroseq Rev2 IDUA Biotin
gccaggacacccactgtatgat

SEQ ID NO: 11 - DNA, 16 bp, Artificial Sequence
  IDUA-Seq2 primer
tggggctcatggccct

SEQ ID NO: 12 - DNA, 48 bp, Artificial Sequence
  pyrosequencing analysis sequence
gttggatggagaacaactctagggcagaggtctcaagaggctggggct

SEQ ID NO: 13 - DNA, 38 bp, Artificial Sequence
  dispensation order
cgtgatgagacactcgtagcagagtctgcagagctgca

(19) United States
(12) Patent Application Publication
(10) Pub. No.: US 2022/0340900 A1
(43) Pub. Date: Oct. 27, 2022

(54) CHEMICALLY MODIFIED OLIGONUCLEOTIDES FOR RNA EDITING

(71) Applicant: PROQR THERAPEUTICS II B.V., Leiden (NL)

(72) Inventors: Janne Juha Turunen, Leiden (NL); Bart Klein, Leiden (NL); Lenka Van Sint Fiet, Leiden (NL); Antti Aalto, Leiden (NL); Cherie Paige Kemmel, Leiden (NL); Tess Hoogeboom, Leiden (NL); Lisanne Alieda Van Wissen, Leiden (NL)

(21) Appl. No.: 17/442,918

(22) PCT Filed: Apr. 2, 2020
(86) PCT No.: PCT/EP2020/059369

§ 371 Date: Sep. 24, 2021

(30) Foreign Application Priority Data
Apr. 3, 2019 (GB) 1904709.1

(51) Int. Cl. C12N 15/113

(52) U.S. Cl. CPC C12N 15/113; C12N 2310/11; C12N 2310/3125; C12N 2310/344; C12N 2310/315; C12N 2310/321; C12N 2310/322; C12N 2310/346

(57) ABSTRACT

The invention relates to single-stranded RNA editing antisense oligonucleotides (AONs) for binding to a target RNA molecule for deaminating a target nucleotide, preferably an adenosine, present in the target RNA molecule and recruiting, in a cell, preferably a human cell, an enzyme with nucleotide deamination activity, preferably an ADAR enzyme, to deaminate the target nucleotide in the target RNA molecule. The AONs carry at least one methylphosphonate-modified internucleosidic linkage on a position that would render the AON more stable in comparison to an AON not carrying that methylphosphonate modification at that position.

### TECHNICAL FIELD

The invention relates to the field of medicine. In particular, it relates to the field of RNA editing, whereby an RNA molecule in a cell is targeted by an antisense oligonucleotide (AON) to specifically change a target nucleotide present in the target RNA molecule. The invention is aimed at amending a specific nucleotide, such as a mutated nucleotide that may cause disease, in the target RNA molecule by engaging an enzyme having deaminase activity. More specifically, the invention relates to AONs that are chemically modified at preferred positions to increase their in vivo and in vitro stability, and thereby increase their RNA editing ability.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called adenosine deaminase and cytidine deaminase, respectively. The most extensively studied RNA editing system is the system involving the adenosine deaminase enzyme.

Adenosine deaminase is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, more or less predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result thereof, exons may be included or skipped. The adenosine deaminases are part of a family of enzymes known as Adenosine Deaminases acting on RNA (ADAR), which include human deaminases hADAR1 and hADAR2, as well as hADAR3. However, for hADAR3 no deaminase activity has been shown yet.

The use of oligonucleotides to edit a target RNA applying adenosine deaminase has been described (e.g. Montiel-Gonzalez et al. PNAS 2013, 110(45):18285-18290; Vogel et al. 2014. Angewandte Chemie Int Ed 53:267-271; Woolf et al. 1995. PNAS 92:8298-8302). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. Clearly, these systems are not readily adaptable for use in humans (e.g. in a therapeutic setting). The oligonucleotides of Woolf et al. (1995) that were 100% complementary to the target RNA sequences, only appeared to function in cell extracts or in Xenopus oocytes by microinjection, and suffered from severe lack of specificity: nearly all adenosines in the target RNA strand that was complementary to the antisense oligonucleotide were edited. An oligonucleotide, 34 nucleotides in length, wherein each nucleotide carried a 2′-O-methyl (2′-OMe) modification, was tested and shown to be inactive in Woolf et al. (1995). In order to provide stability against nucleases, a 34-mer RNA, modified with 2′-OMe and modified with phosphorothioate (PS) linkages at the 5′- and 3′-terminal 5 nucleotides, was also tested. It was shown that the central unmodified region of this oligonucleotide could promote editing of the target RNA by endogenous ADAR, with the terminal modifications providing protection against exonuclease degradation. However, this system did not show deamination of a specific target adenosine in the target RNA sequence. As mentioned, nearly all adenosines opposite an unmodified nucleotide in the antisense oligonucleotide were edited (therefore nearly all adenosines opposite nucleotides in the central unmodified region, if the 5′- and 3′-terminal 5 nucleotides of the antisense oligonucleotide were modified, or nearly all adenosines in the target RNA strand if no nucleotides were modified).

It is known in the art that ADAR may act on any dsRNA. Through a process sometimes referred to as ‘promiscuous editing’, the enzyme will edit multiple A's in the dsRNA. Hence, there is a need for methods and means that circumvent such promiscuous editing and only target specific adenosines in a target RNA molecule to become therapeutic applicable. Vogel et al. (2014) showed that such off-target editing can be suppressed by using 2′-OMe-modified nucleotides in the oligonucleotide at positions opposite to adenosines that should not be edited, and used a non-modified nucleotide directly opposite to the specifically targeted adenosine on the target RNA. However, the specific editing effect at the target nucleotide has not been shown to take place without the use of recombinant ADAR enzymes having covalent bonds with the AON.

WO 2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion there is no need for conjugated entities or presence of modified recombinant ADAR enzymes. WO 2016/097212 describes the recruitment portion as being a stem-loop structure mimicking either a natural substrate (e.g. the GluB receptor) or a Z-DNA structure known to be recognized by the dsRNA binding regions of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described in WO 2016/097212 is an intramolecular stem-loop structure, formed within the AON itself, and able to attract ADAR. WO 2017/220751 and WO 2018/041973 describe AONs that do not comprise such a recruitment portion but that are (almost fully) complementary to the targeted area, except for one or more mismatches, or so-called ‘wobbles’ or bulges. The sole mismatch may be the nucleotide opposite the target adenosine, but in other embodiments AONs were described with multiple bulges and/or wobbles when attached to the target sequence area. It appeared possible to achieve in vitro, ex vivo and in vivo RNA editing with AONs lacking a recruitment portion and with endogenous ADAR enzymes when the sequence of the AON was carefully selected such that it could attract ADAR. The ‘orphan nucleotide’, which is defined as the nucleotide in the AON that is positioned directly opposite the target adenosine in the target RNA molecule, did not carry a 2′-OMe modification. The orphan nucleotide could also be a DNA nucleotide (carrying no 2′ modification is the sugar entity), wherein the remainder of the AON did carry 2′-O-alkyl modifications at the sugar entity (such as 2′-OMe), or the nucleotides within the so-called ‘Central Triplet’ (=the orphan nucleotide with its two direct neighbouring nucleotides within the AON) or directly surrounding the Central Triplet contained particular chemical modifications (or were DNA) that further improved the RNA editing efficiency and/or increased the resistance against nucleases. Such effects could even be further improved by using sense oligonucleotides (SONs) that ‘protected’ the AONs against breakdown (described in WO2018/134301).

Despite the achievements outlined above, there remains a need for improved compounds that can utilise (endogenous) cellular pathways and enzymes that have deaminase activity, such as naturally expressed ADAR enzymes to more specifically and more efficiently edit endogenous nucleic acids in mammalian cells, even in whole organisms, to alleviate disease.

### SUMMARY OF THE INVENTION

The invention relates to an antisense oligonucleotide (AON) capable of forming a double stranded complex with a target nucleic acid molecule in a cell, for use in the deamination of a target nucleotide in the target nucleic acid molecule, preferably an adenosine, wherein the nucleotide in the AON that is directly opposite the target nucleotide is the orphan nucleotide, and wherein the AON comprises one or more methylphosphonate (MP) linkages. The invention therefore provides an AON comprising a sequence configured for the deamination of a target nucleotide in the target nucleic acid molecule, preferably an adenosine. Preferably, the AON is capable of engaging an entity, such as an enzyme, with deamination activity, and preferably the target nucleotide is an adenosine that is deaminated by the deaminating enzyme to an inosine. The internucleotide linkage numbering is such that linkage number 0 is the linkage 5′ from the orphan nucleotide, and wherein the linkage positions in the oligonucleotide are positively (+) incremented towards the 5′ end and negatively incremented towards the 3′ end. Preferably, the AON comprises one or more MP linkages at linkage positions 0, −1, −2, −3, −4, −5 and/or −6, more preferably the AON comprises MP linkages at linkage positions −0 and/or −2. In one embodiment, the AON comprises a single MP linkage. In one embodiment, the orphan nucleoside and/or the nucleoside 3′ of the orphan nucleoside are linked to their respective 3′ neighbouring nucleosides (i.e. at the −1 and −2 linkage positions, respectively) with an MP linkage. Especially preferred is the aspect that the MP linkage renders the AON more stable than an AON lacking that MP linkage when compared in an in vitro stability assay, as outlined in the non-limiting examples herein. In one preferred aspect, the AON of the present invention comprises at least one nucleotide comprising a 2′-OMe or a 2′-MOE ribose modification, and the orphan nucleotide does not carry a 2′-OMe or a 2′-MOE ribose modification.

In one embodiment, the invention relates to a pharmaceutical composition comprising an AON according to the invention, and a pharmaceutically acceptable carrier.

In another embodiment, the invention relates to an AON according to the invention, or a pharmaceutical composition according to the invention, for use in the treatment or prevention of a genetic disorder.

The invention also relates to a method for the deamination of at least one target nucleotide, preferably an adenosine, present in a target RNA molecule in a cell, the method comprising the steps of: providing the cell with an AON or a pharmaceutical composition according to the invention; allowing annealing of the AON to the target RNA molecule; allowing a mammalian enzyme with nucleotide deaminase activity to deaminate the target nucleotide in the target RNA molecule; and optionally identifying the presence of the deaminated nucleotide in the target RNA molecule.

The invention also relates to a method for the deamination of at least one target nucleotide, preferably an adenosine, present in a target RNA molecule, the method comprising the steps of: providing an AON according to the invention; allowing annealing of the AON to the target RNA molecule; allowing a mammalian enzyme with nucleotide deaminase activity to deaminate the target nucleotide in the target RNA molecule; and identifying the presence of the deaminated nucleotide in the target RNA molecule.

### BRIEF DESCRIPTION OF THE DRAWINGS

One or more embodiments of the invention will now be described, by way of example only, with reference to the accompanying drawings, in which:

FIG. 1 shows the methylphosphonate (MP) modification structure, linking two DNA nucleotides.

FIG. 2 shows the sequence of the target mouse IDUA gene (upper strand in both panels) from 5′ to 3′ (SEQ ID NO:1) with the target adenosine popping upwards. Below the target sequence the sequence is given (3′ to 5′) of the ADAR102-1 (upper panel) and the ADAR102-13 (lower panel) antisense oligonucleotides (both SEQ ID NO:2) with the nucleotide opposite the target adenosine popping downwards. The two nucleotides in the oligonucleotides that are given in capitals are DNA. The \hat{ } under the two nucleotides in ADAR102-13 represent the presence of an MP linkage modification connecting these nucleosides with their respective 3′ neighbouring nucleosides. Asterisks represent the four phosphorothioate (PS) linkage modifications between the ultimate five nucleosides of the AONs on each end.

FIG. 3 shows the results of a stability assay in which four AONs were subjected to a mix of endo- and exonucleases in a biochemical assay, and in which their stability was monitored over time (0, 30 and 120 minutes). Whereas the AONs comprising DNA nucleotides and no linkage modification (ADAR102-1) and comprising DNA nucleotides linked by a PS linkage (ADAR102-25) disappeared after 30 min incubation, the AON carrying two DNA nucleotides linked to each other by a MP modified linkage (ADAR102-13) appeared similarly stable to an AON carrying 2′-OMe modifications (instead of 2′-H) in the sugar moiety plus a PS modification between the two nucleotides opposite the target adenosine in a mouse IDUA RNA target molecule.

FIG. 4 shows the percentage of RNA editing, over a period of 1 hr, obtained with ADAR102-1 and ADAR102-13.

FIG. 5 shows nucleotide and linkage numbering applied to an AON sequence. The example AON sequence is for a portion of the IDUA sequence given in FIG. 2. This specific sequence is only provided as an example for the purpose of demonstrating the nucleotide and linkage numbering system. Below the nucleotide sequence the order of nucleotides is given in which the “0” position refers to the orphan nucleotide (i.e. opposite the target adenosine that is to be deaminated in the target sequence). Towards the 5′ end of the AON the nucleotide numbering is increasing to +19, whereas towards the 3′ end of the AON the nucleotide numbering is decreasing to −12. Hence in this example there are 32 nucleotides shown (of a potentially longer AON). The linkage numbering is different, with the linkage 5′ of a nucleotide corresponding to the nucleotide number. The linkage numbering is given in the top row. As can be seen, the linkage referred to as “0” in the top row is the linkage 5′ of nucleotide “0”, with increasing linkage numbering towards the 5′ end to +19, and with decreasing linkage numbering towards the 3′ end to −12. It is to be understood that the ultimate ‘linkage’ +19 is not linked in this particular example but may be linked to a next nucleotide in any given AON if it is longer at the 5′ terminus. The same is true for the 3′ end, where additional nucleotides may be attached.

FIG. 6 shows the results of an editing assay conducted with AONs having no MP modification and MP modifications at linkage positions 0, −1, −2 and −3. Results are from two independent experiments, with the average editing activity and standard deviation shown.

FIG. 7 shows the results of an editing assay conducted with AONs having no MP modification and MP modifications at linkage positions −4, −5 and −6. Results are from one editing experiments.

FIG. 8 shows the results of an in cell editing assay conducted with an AON having no MP modifications compared with an AON having MP modifications at the indicated linkage positions.

### DETAILED DESCRIPTION

There is a constant need for improving the pharmacokinetic properties of RNA-editing antisense oligonucleotides (AONs, sometimes referred to as ‘editing oligonucleotides’, or ‘EONs’) without negatively affecting editing efficiency of the target adenosine in the target RNA. Many chemical modifications exist in the generation of AONs, whose properties are not always compatible with the desire of achieving efficient RNA editing. In the search for better pharmacokinetic properties, it was found earlier that a 2′-O-methoxyethyl (or 2′-methoxyethoxy, or 2′-MOE) modification of the ribose of some, but not all, nucleotides surprisingly appeared compatible with efficient ADAR engagement and editing (WO 2019/158475). In a similar fashion, it was found earlier that a phosphorothioate (PS) linkage at some, but not all, internucleotide linkages surprisingly appeared compatible with efficient ADAR engagement and editing (WO 2019/219581). Also, it was found earlier that phosphonoacetate linkage modifications and/or unlocked nucleic acid (UNA) ribose modifications of some, but not all, positions in the AON appeared compatible with efficient engagement of an enzyme with nucleotide deamination activity and with subsequent deamination (PCT/EP2020/053283, unpublished). Whereas the properties of phosphonoacetate and UNA modifications were known as such, the compatibility thereof with engagement of enzymes with nucleotide deamination activity and with the deamination reaction was not known.

The inventors of the present invention have now unravelled that the introduction of a particular type of internucleoside linkage modification, wherein the orphan nucleoside in the AON is linked by this particular modified linkage to its 3′ neighbouring nucleoside, increased the stability of the entire AON in a significant manner, while the AON appeared still capable of engaging an enzyme with deamination activity and, importantly, appeared to be capable of causing deamination of the target nucleotide in the target RNA molecule. These findings can, in principle, be used with any form of base editing employing synthetic oligonucleotides involving, for example, ADAR or ADAR deaminase domains, be they natural or recombinant, truncated or full length, fused to other proteins or not (e.g. Stafforst and Schneider, 2012, Angew Chem Int 51:11166-11169; Schneider et al. 2014, Nucleic Acids Res 42:e87; Montiel-Gonzalez et al. 2016, Nucleic Acids Res 44:e157). The skilled person is aware of a variety of enzymes that have nucleotide deaminase activity, such as ADAR1, ADAR2, APOBEC, or fusion proteins containing the active domains of these enzymes fused to other proteins for targeted RNA binding, and the like. The invention in particular relates to AONs comprising a methylphosphonate (MP) internucleosidic linkage modification. The internucleotide linkage numbering is such that linkage number 0 is the linkage 5′ from the orphan nucleotide, and wherein the linkage positions in the oligonucleotide are positively (+) incremented towards the 5′ end and negatively incremented towards the 3′ end. In terms of the nucleotide numbering, the orphan nucleotide is position 0 and the nucleotide numbers are positively (+) incremented towards the 5′ end and negatively incremented towards the 3′ end. In a particular embodiment of the invention, the AON comprises one or more MP linkages at linkage positions 0, −1, −2, −3, −4, −5 and/or −6. At these positions, the MP linkage is particularly effective at stabilising the AON without preventing editing activity. In one embodiment, the AON comprises 7, 6, 5, 4, 3, 2 or 1 MP linkages at linkage positions 0, −1, −2, −3, −4, −5 and/or −6. In a preferred embodiment, the AON comprises MP linkages at linkage positions 0, −1, −2, −3, −4 and/or −5, linkage positions 0, −1, −2, −3 and/or −4, linkage positions 0, −1, −2 and/or −3, linkage positions 0, −1 and/or −2. In a particularly preferred embodiment, the AON comprises MP linkages at positions 0 and/or −2. In one embodiment the AON comprises MP linkages between the orphan nucleoside (that is opposite the target adenosine) and the next nucleoside in the AON at the 3′ side (i.e. the −1 linkage position). In one embodiment, the AON comprises MP linkages at all linkage positions. In another embodiment, the AON comprises a single MP linkage. In a particular embodiment, the AON is selected from: an AON comprising an MP linkage at only position 0, an AON comprising an MP linkage only at linkage position −1, an AON comprising an MP linkage only at linkage position −2, an AON comprising an MP linkage only at linkage position −3, an AON comprising an MP linkage only at linkage position −4, an AON comprising an MP linkage only at linkage position −5, an AON comprising an MP linkage only at linkage position −6, and an AON comprising an MP linkage only at linkage positions −2 and −4.

This now solves the problem of achieving a higher stability of the AON, while maintaining the ability of it to engage an enzyme with nucleotide deamination activity and obtain RNA editing of a target adenosine in a target RNA molecule.

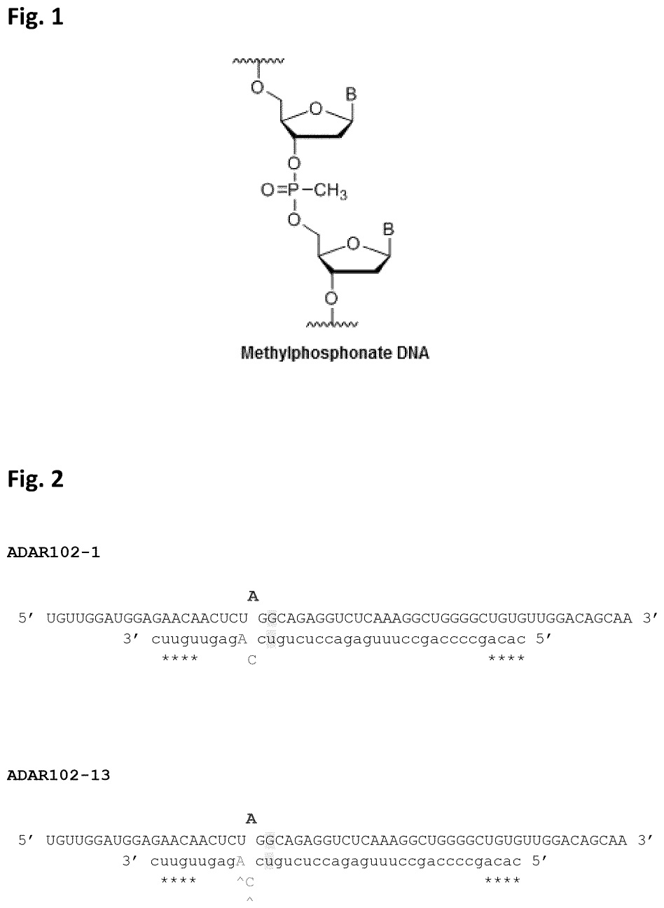

The use of MP for stabilizing AONs is not a new concept (see e.g. Agrawal et al. 1997. Proc Natl Acad Sci USA 94:2620-2625), but, in view of what was previously found with modifications within and surrounding the nucleotide in an RNA editing AON opposite the target nucleotide in the target RNA molecule, it was highly surprising to find that having an MP modification at the linkage connecting the orphan nucleoside with a neighbouring nucleoside, preferably its 3′ neighbouring nucleoside, did still allow RNA editing, as shown herein. It should be understood, that the invention encompasses any AON that can bind to a target RNA molecule, recruit any protein (naturally expressed proteins as well as foreign proteins, including fusion proteins of different or the same origin) with nucleotide (including adenosine) deamination activity, as long as at least one internucleosidic linkage comprises a MP linkage, preferably wherein the MP is present in the internucleosidic linkage connecting the AON nucleoside which is opposite the target nucleotide in the target RNA molecule, with its 3′ neighbouring nucleoside, and wherein the 3′ neighbouring nucleoside can further be linked by another MP linkage to its respective 3′ neighbouring nucleoside. Hence, the present invention relates to an AON comprising nucleotides that are linked by internucleosidic linkages, wherein the AON—when forming a double stranded nucleic acid structure by binding to a complementary target nucleic acid sequence—is capable of recruiting an enzyme with nucleotide deaminase activity on a target nucleotide in the complementary target nucleic acid sequence, characterized in that the AON has been optimized for stability by the introduction of at least one MP-modified internucleosidic linkage. The chemical structure of the MP linkage (here between DNA nucleosides) is shown in FIG. 1. An important role of this modification is to protect the polymer from nuclease-mediated degradation. In one aspect of the invention, at least one internucleoside linkage, when not being modified by MP, may be an unmodified phosphodiester linkage. The MP modification may also exist in addition to modifications to the ribose 2′ group. The ribose 2′ groups in the AON can be independently selected from 2′-H (i.e. DNA), 2′-OH (i.e. RNA), 2′-OMe, 2′-MOE, 2′-F, or 2′-4′-linked (i.e. a locked nucleic acid or LNA), or other 2′ substitutions as further outlined below. The 2′-4′ linkage can be selected from linkers known in the art, such as a methylene linker or constrained ethyl linker. In all cases, the modifications should be compatible with editing such that the oligonucleotide fulfils its role as an RNA editing AON. The AON may be further optimized for binding to an enzyme with nucleotide deamination activity by generating at least one unlocked nucleic acid (UNA) ribose modification in a position which is not incompatible with editing activity of the enzyme having nucleotide deaminase activity. In a UNA modification, there is no carbon-carbon bond between the ribose 2′ and 3′ carbon atoms. UNA ribose modifications therefore increase the local flexibility in oligonucleotides. UNAs can lead to effects such as improved pharmacokinetic properties through improved resistance to degradation. UNAs can also decrease toxicity and may participate in reducing off-target effects. A UNA ribose modification should preferably be avoided at the orphan nucleotide as disruption of binding with the enzyme with nucleotide deaminase activity would be significant (GB 1901873.8, unpublished). The UNA ribose modification may be the only ribose modification in the AON, but the UNA modification may exist in addition to modifications to the ribose 2′ group, either at positions different to the UNA modifications or at the same positions as the UNA modifications. The ribose 2′ groups in the AON can be independently selected from 2′-H (i.e. DNA), 2′-OH (i.e. RNA), 2′-OMe, 2′-MOE, 2′-F, or 2′-4′-linked (i.e. a locked nucleic acid or LNA), or other 2′ substitutions as further outlined below. The 2′-4′ linkage can be selected from linkers known in the art, such as a methylene linker or constrained ethyl linker. Different 2′ modifications are discussed in further detail in WO2016/097212, WO2017/220751, WO2018/041973, WO2018/134301, GB1808146.3 (unpublished), GB 1901873.8 (unpublished), and PCT/EP2019/053291 (unpublished). In all cases, the modifications should be compatible with editing such that the oligonucleotide fulfils its role as an RNA editing AON. In all aspects of the invention, the enzyme with nucleotide deaminase activity is preferably ADAR1 or ADAR2. In a highly preferred embodiment, the AON is an RNA editing single-stranded AON that targets a pre-mRNA or an mRNA, wherein the target nucleotide is preferably an adenosine in the target RNA, wherein the adenosine is deaminated to an inosine, which is being read as a guanosine by the translation machinery. In a further preferred embodiment, the adenosine is located in a UGA or UAG stop codon, which is edited to a UGG codon; or wherein two target nucleotides are the two adenosines in a UAA stop codon, which codon is edited to a UGG codon through the deamination of both target adenosines, wherein two nucleotides in the oligonucleotide mismatch with the target nucleic acid.

The AON according to the invention can comprise internucleoside linkage modifications other than, or in addition to, the MP linker modifications. In one embodiment one such other internucleoside linkage can be a phosphonoacetate modified linkage. In another embodiment, the internucleotide linkage can be a phosphodiester wherein the OH group of the phosphodiester has been replaced by alkyl, alkoxy, aryl, alkylthio, acyl, —NR1R1, alkenyloxy, alkynyloxy, alkenylthio, alkynylthio, —S-Z+, —Se-Z+, or —BH3-Z+, and wherein R1 is independently hydrogen, alkyl, alkenyl, alkynyl, or aryl, and wherein Z+ is ammonium ion, alkylammonium ion, heteroaromatic iminium ion, or heterocyclic iminium ion, any of which is primary, secondary, tertiary or quaternary, or Z is a monovalent metal ion, and is preferably a phosphorothioate (PS) linkage.

In the AON of the present invention, the orphan nucleotide generally comprises a ribose with a 2′-OH group, or a deoxyribose with a 2′-H group, and preferably does not comprise a ribose carrying a 2′-OMe modification. Further, the AON of the present invention generally does not comprise 2′-MOE modifications at certain positions relative to the orphan nucleotide, and further does comprise 2′-MOE modifications at other positions within the AON. The AONs of the present invention preferably do not comprise a recruitment portion as described in WO 2016/097212. The AONs of the present invention preferably do not comprise a portion that is capable of forming an intramolecular stem-loop structure. The AON does preferably not include a 5′-terminal 06-benzylguanine modification. The AON preferably does not include a 5′-terminal amino modification. The AON is preferably not covalently linked to a SNAP-tag domain.

The invention relates to a method for the deamination of at least one target adenosine present in a target RNA molecule in a cell, the method comprising the steps of providing the cell with an AON according to a first aspect of the invention, or a composition according to a second aspect of the invention, allowing uptake by the cell of the AON, allowing annealing of the AON to the target RNA molecule, allowing a mammalian enzyme with nucleotide deaminase activity to deaminate the target nucleotide in the target RNA molecule, and optionally identifying the presence of the deaminated nucleotide in the target RNA molecule. Preferably, the presence of the target RNA molecule is detected by either (i) sequencing the target sequence, (ii) assessing the presence of a functional, elongated, full length and/or wild type protein when the target adenosine is located in a UGA or UAG stop codon, which is edited to a UGG codon through the deamination, (iii) assessing the presence of a functional, elongated, full length and/or wild type protein when two target adenosines are located in a UAA stop codon, which is edited to a UGG codon through the deamination of both target adenosines, (iv) assessing whether splicing of the pre-mRNA was altered by the deamination; or (v) using a functional read-out, wherein the target RNA after the deamination encodes a functional, full length, elongated and/or wild type protein. The present invention therefore also relates to AONs that target premature termination stop codons (PTCs) present in the (pre)mRNA to alter the adenosine present in the stop codon to an inosine (read as a G), which in turn then results in read-through during translation and a full length functional protein. In one particular embodiment, the present invention relates to AONs for use in the treatment of cystic fibrosis (CF), and in an even further preferred embodiment, the present invention relates to AONs for use in the treatment of CF wherein PTCs such as the G542X (UGAG), W1282X (UGAA), R553X (UGAG), R1162X (UGAG), Y122X (UAA, both adenosines), W1089X, W846X, and W401X mutations are modified through RNA editing to amino acid encoding codons, and thereby allowing the translation to full length proteins. The teaching of the present invention, as outlined herein, is applicable for all genetic diseases that may be targeted with AONs and may be treated through RNA editing. One other particular preferred example is Usher syndrome type II caused by mutations in the USH2A gene.

In one aspect, the invention relates to an AON capable of forming a double stranded complex with a target RNA molecule in a cell, for use in the deamination of a target adenosine in a disease-related splice mutation present in the target RNA molecule, wherein the orphan nucleotide in the AON (that is opposite the target adenosine) does not carry a 2′-OMe modification; wherein the nucleotide directly 5′ and/or 3′ from the orphan nucleotide (which nucleotides—together with the orphan nucleotide—form the Central Triplet) carry a sugar modification and/or a base modification to render the AON more stable and/or more effective in RNA editing; and wherein at least one linkage in the AON, preferably within the Central Triplet, carries an MP modification. In a preferred embodiment, at least one internucleoside linkage connecting two nucleosides in the Central Triplet carries an MP modification. In another preferred aspect the orphan nucleotide is DNA, and in an even more preferred aspect, the orphan nucleotide as well as the nucleotide 5′ and/or 3′ of the nucleotide opposite the target adenosine are DNA nucleotides, while the remainder (not DNA) of the nucleotides in the AON are preferably 2′-O-alkyl modified ribonucleotides. When two nucleotides are DNA all others may be RNA and may be 2′-OMe or 2′-MOE modified, whereas in particular aspects the third nucleotide in the Central Triplet opposite the target adenosine may be RNA and non-modified, as long as the nucleotide opposite the target adenosine is not 2′-OMe modified. In a preferred embodiment, the AON according to the invention comprises 2, 3, 4, 5, 6, 7, 8, 9 or 10 mismatches, wobbles and/or bulges with the complementary target RNA region. Preferably, the nucleotide that is opposite the target adenosine is a cytidine, a deoxycytidine, a uridine, a deoxyuridine, or is abasic. When the nucleotide opposite the target adenosine is a cytidine or a deoxycytidine, the AON comprises at least one mismatch with the target RNA molecule. When the nucleotide opposite the target adenosine is a uridine or a deoxyuridine, the AON may be 100% complementary and not have any mismatches, wobbles or bulges in relation to the target RNA. However, in a preferred aspect one or more additional mismatches, wobbles and/or bulges are present between AON and target RNA whether the nucleotide opposite the target adenosine is a cytidine, a deoxycytidine, a uridine, or a deoxyuridine. In another preferred embodiment, the nucleotide directly 5′ and/or 3′ from the orphan nucleotide (together with the orphan nucleotide forming the Central Triplet) comprises a ribose with a 2′-OH group, or a deoxyribose with a 2′-H group, or a mixture of these two. The Central Triplet then consists of DNA-DNA-DNA; DNA-DNA-RNA; DNA-RNA-DNA; DNA-RNA-RNA; RNA-DNA-DNA; RNA-DNA-RNA; RNA-RNA-DNA; or RNA-RNA-RNA; wherein the middle nucleotide does not have a 2′-OMe modification (when RNA) and either or both surrounding nucleotides also do not have a 2′-OMe modification. It is then preferred that all other nucleotides in the AON then do have a 2′-O-alkyl group, preferably a 2′-OMe group, or a 2′-MOE group, or any modification as disclosed herein. The AONs of the present invention comprise at least one MP linkage modification. Wobbles, mismatches and/or bulges of the AON of the present invention with the target sequence do not prevent hybridization of the oligonucleotide to the target RNA sequence, but add to the RNA editing efficiency by the ADAR present in the cell, at the target adenosine position. The person skilled in the art is able to determine whether hybridization under physiological conditions still does take place. The AON of the present invention can recruit (engage) a mammalian ADAR enzyme present in the cell, wherein the ADAR enzyme comprises its natural dsRNA binding domain as found in the wild type enzyme. The AONs according to the present invention can utilise endogenous cellular pathways and naturally available ADAR enzyme, or enzymes with ADAR activity (which may be yet unidentified ADAR-like enzymes) to specifically edit a target adenosine in a target RNA sequence. As disclosed herein, the single-stranded AONs of the invention are capable of deamination of a specific target, such as adenosine, in a target RNA molecule. Ideally, only one nucleotide is deaminated. Alternatively, 1, 2, or 3 further nucleotides are deaminated, but preferably only one. The AONs of the invention can be designed for and used with a variety of nucleotide deaminase enzymes. A particular example is ADAR. Taking ADAR as an example, the ADAR can be naturally expressed or produced artificially (e.g. by recombinant expression or protein synthesis). The ADAR can be wild-type or modified. Taking the features of the AONs of the present invention together, there is no need for modified recombinant ADAR expression. The AONs of the invention are not particularly limited with regard to conjugated entities attached to the AON. However, there is no need for conjugated entities attached to the AON. As such, AONs lacking conjugated entities attached to the AON form a preferred embodiment. The AONs of the invention are not particularly limited regarding recruitment portions that are not complementary to the target RNA sequence. However, there is no need for the presence of long recruitment portions that are not complementary to the target RNA sequence. Consequently, AONs lacking long recruitment portions that are not complementary to the target RNA sequence form a preferred embodiment. Besides that, the AON of the present invention does allow for the specific deamination of a target nucleotide present in the target nucleic acid molecule by a natural nucleotide deaminase enzyme comprising a natural dsRNA binding domain as found in the wild type enzyme, without the risk of promiscuous editing elsewhere in the RNA/AON complex.

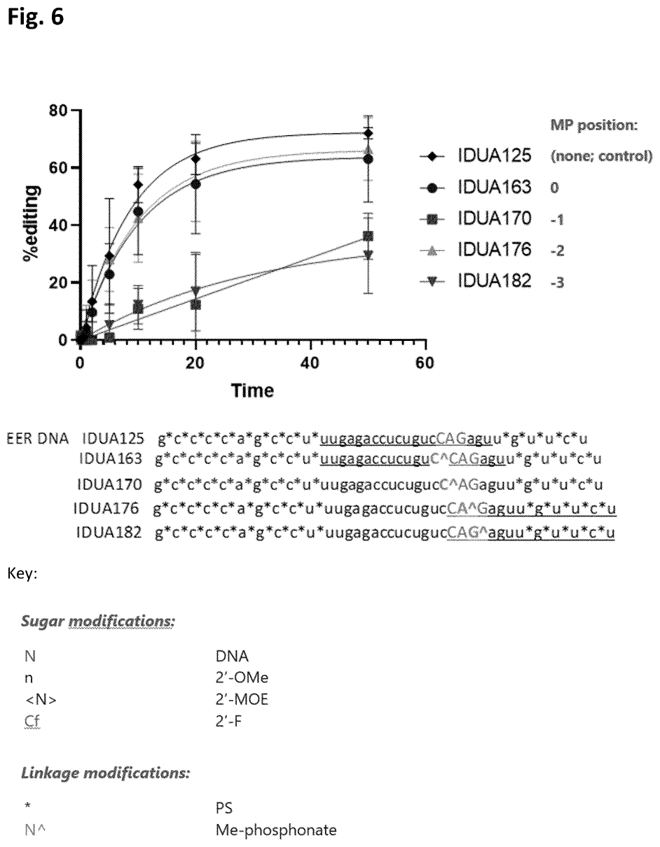

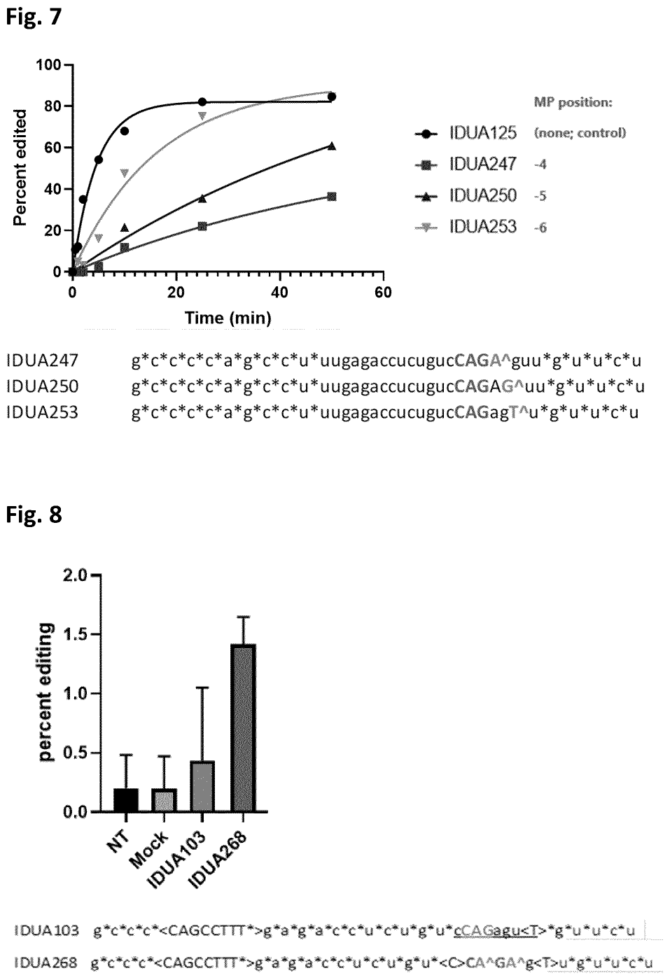

The invention relates to an antisense oligonucleotide (AON) capable of forming a double stranded complex with a target nucleic acid molecule in a cell, for use in the deamination of a target nucleotide in the target nucleic acid molecule, preferably an adenosine, wherein the nucleotide in the AON that is directly opposite the target nucleotide is the orphan nucleotide, and wherein the AON comprises one or more methylphosphonate (MP) linkages. The AON of the invention is capable of engaging an entity, preferably an enzyme, with deamination activity that is preferably endogenously present in a cell, preferably a mammalian, more preferably a human cell, to provide deamination of the target nucleotide in the target nucleic acid molecule. A preferred target nucleic acid molecule is an RNA molecule. The double stranded AON/target nucleic acid molecule complex interacts through Watson-Crick base-pairing. Preferably, the orphan nucleotide does not carry a 2′-OMe or 2′-MOE ribose modification. Preferably, the orphan nucleoside and/or the nucleoside 3′ of the orphan nucleoside are linked to their respective 3′ neighbouring nucleosides with an MP linkage (i.e. at the −1 and −2 positions). More preferably, both the orphan nucleoside and its 3′ neighbouring nucleoside are linked through MP modified linkages with their respective 3′ neighbouring nucleosides, as exemplified by AON ADAR102-13 (FIG. 2). In a particular embodiment of the invention, the AON comprises one or more MP linkages at linkage positions 0, −1, −2, −3, −4, −5 and/or −6, as exemplified by IDUA163, IDUA170, IDUA176, IDUA182, IDUA247, IDUA250 and IDUA254 (FIGS. 6 and 7). In one embodiment, the AON comprises 7, 6, 5, 4, 3, 2 or 1 MP linkages at linkage positions 0, −1, −2, −3, −4, −5 and/or −6. In a preferred embodiment, the AON comprises MP linkages at linkage positions 0, −1, −2, −3, −4 and/or −5, linkage positions 0, −1, −2, −3 and/or −4, linkage positions 0, −1, −2 and/or −3, linkage positions 0, −1 and/or −2. In a particularly preferred embodiment, the AON comprises MP linkages at positions 0 and/or −2 as exemplified by IDUA163 and IDUA176 (FIG. 6). In one embodiment, the AON comprises MP linkages at all linkage positions. In another embodiment, the AON comprises a single MP linkage. In a particular embodiment, the AON is selected from: an AON comprising an MP linkage at only position 0 (as exemplified by IDUA163), an AON comprising an MP linkage only at linkage position −1 (as exemplified by IDUA170), an AON comprising an MP linkage only at linkage position −2 (as exemplified by IDUA176 and IDUA264), an AON comprising an MP linkage only at linkage position −3 (as exemplified by IDUA182), an AON comprising an MP linkage only at linkage position −4 (as exemplified by IDUA247), an AON comprising an MP linkage only at linkage position −5 (as exemplified by IDUA250), an AON comprising an MP linkage only at linkage position −6 (as exemplified by IDUA253), and an AON comprising an MP linkage only at linkage positions −2 and −4 (as exemplified by IDUA267 and IDUA268). In a preferred embodiment, the MP linkage connects a DNA nucleoside with another nucleoside. In another preferred embodiment, the MP linkage connects a DNA nucleoside with a DNA nucleoside. In one important aspect of the invention, the MP linkage renders the AON more stable than an AON lacking that MP linkage when compared in an in vitro stability assay. The skilled person, based on the current teaching, will be able to determine whether an MP modification, as exemplified herein, renders the AON more stable than an AON lacking an MP modification at a certain selected position in the AON, by using the timed in vitro stability assay disclosed by the inventors of the present invention (applying a Nucleases Mix comprising Phosphodiesterase I from Crotalus adamenteus venom, DNase I, RNase A and Nuclease BAL-31 mixed in Nuclease buffer and nuclease-free water), which is used as a non-limiting example of how stability can be tested in the laboratory. It is also an important aspect of the invention that the AON comprising at least one MP modified linkage, rendering the AON more stable than an AON lacking the MP modification at that position, is able to engage an entity with deamination activity, such as a deamination enzyme, preferably an ADAR enzyme, to achieve RNA editing of the target nucleotide that is present in the target nucleic acid molecule. Hence, in a preferred embodiment, the ability of the AON to provide deamination of the target nucleotide is preserved to a level of at least 10%, at least 20%, at least 30%, at least 40%, at least 50%, or at least 60% in comparison to an AON lacking the one or more MP linkages. Like the stability assay, the skilled person is able, based on the current teaching, to determine the level of capability to achieve RNA editing and compare this to an AON lacking the MP linkage at the specified position. In a preferred aspect, the AON further comprises at least one phosphorothioate (PS) or a phosphonoacetate internucleotide linkage, and/or at least one nucleotide comprising an unlocked nucleic acid (UNA) ribose modification. In a more preferred aspect PS linkages are present at both termini of the AON, connecting the ultimate five nucleosides on each end. In another preferred aspect, the AON of the invention further comprises one or more nucleotides comprising a substitution at the 2′ position of the ribose, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy (2′-MOE); -dimethylamino oxyethoxy; and -dimethylaminoethoxyethoxy. In an even more preferred embodiment, the AON of the present invention comprises at least one nucleotide comprising a 2′-OMe or a 2′-MOE ribose modification, and wherein the orphan nucleotide does not carry a 2′-OMe or a 2′-MOE ribose modification. Most preferably, the AON of the present invention is capable in a cell to engage an entity, preferably an enzyme with deaminase activity, preferably an enzyme with adenosine deaminase activity, such as ADAR1 or ADAR2, more preferably ADAR2. Preferably, the enzyme in the cell with deaminase activity is a naturally occurring (and endogenously present) human deaminase enzyme. The AON according to the present invention is preferably at least 10, 11, 12, 13, 14, 15, 16, 17, 18, 19, 20, 21, 22, 23, 24, 25, 26, 27, 28, 29, 30, 31, or 32 nucleotides in length. Also, preferably, the AON is shorter than 100 nucleotides, more preferably shorter than 60 nucleotides.

In another embodiment, the invention relates to a pharmaceutical composition comprising the AON according to the invention, and a pharmaceutically acceptable carrier. Pharmaceutically acceptable carriers are known to the person skilled in the art.

In yet another embodiment, the invention relates to an AON according to the invention, or a pharmaceutical composition according to the invention, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, B-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, (Dystrophic) Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber Congenital Amaurosis (such as LCA10), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt disease, Tay-Sachs Disease, Usher syndrome (such as Usher syndrome type I, type II, and type Ill), X-linked immunodeficiency, Sturge-Weber Syndrome, and cancer.

In another embodiment, the invention relates to the use of an AON according to the invention in the manufacture of a medicament for the treatment of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, B-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, (Dystrophic) Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber Congenital Amaurosis (such as LCA10), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt disease, Tay-Sachs Disease, Usher syndrome (such as Usher syndrome type I, type II, and type III), X-linked immunodeficiency, Sturge-Weber Syndrome, and cancer.

In yet another embodiment, the invention relates to a method for the deamination of at least one target nucleotide, preferably an adenosine, present in a target nucleic acid molecule, preferably an RNA target molecule, in a cell, the method comprising the steps of: providing the cell with an AON according to the invention, or the pharmaceutical composition according to the invention; allowing annealing of the AON to the target nucleic acid molecule; allowing a mammalian enzyme with nucleotide deaminase activity to deaminate the target nucleotide in the target nucleic acid molecule; and optionally identifying the presence of the deaminated nucleotide in the target nucleic acid molecule. The mammalian enzyme with nucleotide deaminase activity that is engaged through the use of the AON according to the invention is preferably an adenosine deaminase enzyme, and is capable of altering the target nucleotide in the target nucleic acid molecule, which target nucleotide is then preferably an adenosine that is deaminated to an inosine. The optional step of identifying the presence of the deaminated nucleotide is preferably performed by: sequencing a region of the target nucleic acid molecule, wherein the region comprises the deaminated target nucleotide; assessing the presence of a functional, elongated, full length and/or wild type protein when the target nucleotide is an adenosine located in a UGA or UAG stop codon, which is edited to a UGG codon through the deamination; assessing the presence of a functional, elongated, full length and/or wild type protein when two target adenosines are located in a UAA stop codon, which is edited to a UGG codon through the deamination of both target adenosines; assessing, when the target RNA molecule is pre-mRNA, whether splicing of the pre-mRNA was altered by the deamination; or using a functional read-out, wherein the target nucleic acid molecule after the deamination encodes a functional, full length, elongated and/or wild type protein.

In yet another embodiment, the invention relates to a method for the deamination of at least one target nucleotide, preferably an adenosine, present in a target nucleic acid molecule, preferably a target RNA molecule, the method comprising the steps of: providing an AON according to the invention; allowing annealing of the AON to the target nucleic acid molecule to form a double stranded nucleic acid complex; allowing a mammalian enzyme with nucleotide deaminase activity to deaminate the target nucleotide in the target nucleic acid molecule; and identifying the presence of the deaminated nucleotide in the target nucleic acid molecule.

In another embodiment, the invention relates to a method of treating a subject, preferably a human subject in need thereof, wherein the subject suffers from a genetic disorder caused by a mutation involving the appearance of an adenosine (for instance in a PTC), and in which deamination of that adenosine to an inosine would alleviate, prevent, or ameliorate the disease, comprising the steps of administering to the subject an AON or pharmaceutical composition according to the invention, allowing the formation of a double stranded nucleic acid complex of the AON with its specific complementary target nucleic acid in a cell in the subject; allowing the engagement of an endogenous present enzyme with deamination activity, such as hADAR1 or hADAR2; and allowing the enzyme to deaminate the target adenosine in the target nucleic target molecule to an inosine, thereby alleviating, preventing or ameliorating the genetic disease. The genetic diseases that may be treated according to this method are preferably, but not limited to the genetic diseases listed herein (see above).

### Definitions

The terms ‘adenine’, ‘guanine’, ‘cytosine’, ‘thymine’, ‘uracil’ and ‘hypoxanthine’ (the nucleobase in inosine) as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group), an unlocked nucleic acid (UNA), a nucleotide including a linker comprising a phosphodiester, phosphonoacetate, phosphotriester, phosphorothioate (PS), phosphoro(di)thioate, methylphosphonate (MP), phosphoramidate linkers, and the like. Sometimes the terms adenosine and adenine, guanosine and guanine, cytidine and cytosine, uracil and uridine, thymine and thymidine/uridine, inosine and hypo-xanthine, are used interchangeably to refer to the corresponding nucleobase on the one hand, and the nucleoside or nucleotide on the other. Sometimes the terms nucleobase, nucleoside and nucleotide are used interchangeably, unless the context clearly requires differently, for instance when a nucleoside is linked to a neighbouring nucleoside and the linkage between these nucleosides is modified. In this case, it may be considered that the nucleoside has a modified linker, or that the nucleotide is a modified nucleotide. As stated above, a nucleotide is a nucleoside +one or more phosphate groups. The terms ‘ribonucleoside’ and ‘deoxyribonucleoside’, or ‘ribose’ and ‘deoxyribose’ are as used in the art. Whenever reference is made to an ‘antisense oligonucleotide’, ‘oligonucleotide’, or ‘AON’ both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I.

In a preferred aspect, the AON of the present invention is an oligoribonucleotide that may comprise chemical modifications and may include deoxynucleotides (DNA) at certain specified positions. Terms such as oligonucleotide, oligo, ON, oligonucleotide composition, antisense oligonucleotide, AON, (RNA) editing oligonucleotide, EON, and RNA (antisense) oligonucleotide may be used herein interchangeably. Whenever reference is made to nucleotides in the oligonucleotide construct, such as cytosine, 5-methylcytosine, 5-hydroxymethylcytosine and p-D-Glucosyl-5-hydroxy-methylcytosine are included; when reference is made to adenine, N6-Methyladenine and 7-methyladenine are included; when reference is made to uracil, dihydrouracil, 4-thiouracil and 5-hydroxymethyluracil are included; when reference is made to guanine, 1-methylguanine is included. Whenever reference is made to nucleosides or nucleotides, ribofuranose derivatives, such as 2′-desoxy, 2′-hydroxy, and 2′-O -substituted variants, such as 2′-O-methyl, are included, as well as other modifications, including 2′-4′ bridged variants. Whenever reference is made to oligonucleotides, linkages between two mono-nucleotides may be phosphodiester linkages as well as modifications thereof, including, phosphonoacetate, phosphodiester, phosphotriester, PS, phosphoro(di)thioate, MP, phosphor-amidate linkers, and the like.

The term ‘comprising’ encompasses ‘including’ as well as ‘consisting’, e.g. a composition ‘comprising X’ may consist exclusively of X or may include something additional, e.g. X+Y. The term ‘about’ in relation to a numerical value x is optional and means, e.g. x+10%. The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the AON hybridizes under physiological conditions to the target sequence. The term does not mean that each and every nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence such that the cellular RNA editing enzymes can edit the target adenosine. The term “substantially complementary” therefore also means that in spite of the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, as long as under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. Mismatched nucleotides are G-A, C-A, U-C, A-A, G-G, C-C, U-U pairs. In some embodiments AONs of the present invention comprise fewer than four mismatches, for example 0, 1 or 2 mismatches. Wobble base pairs are: G-U, I-U, I-A, and I-C base pairs.

An AON of the present invention comprises a nucleotide that is directly opposite the target nucleotide present in the target RNA molecule. The nucleotide in the AON that is directly opposite the target nucleotide is herein defined as the ‘orphan nucleotide’. The ‘Central Triplet’ is defined as the region within the AON consisting of the orphan nucleotide plus its 3′ and 5′ neighbouring nucleotides (hence, the Central Triplet=three nucleotides with the orphan nucleotide in the middle).

The term ‘splice mutation’ relates to a mutation in a gene that encodes for a pre-mRNA, wherein the splicing machinery is dysfunctional in the sense that splicing of introns from exons is disturbed and due to the aberrant splicing the subsequent translation is out of frame resulting in premature termination of the encoded protein. Often such shortened proteins are degraded rapidly and do not have any functional activity, as discussed herein. The exact mutation does not have to be the target for the RNA editing; it may be that a neighbouring or nearby adenosine in the splice mutation is the target nucleotide, which conversion to I fixes the splice mutation back to a normal state. The skilled person is aware of methods to determine whether or not normal splicing is restored, after RNA editing of the adenosine within the splice mutation site or area.

An AON according to the present invention may be chemically modified at almost its entirety of nucleosides, for example by providing nucleosides with a 2′-O-methylated sugar moiety (2′-OMe) and/or with a 2′-O-methoxyethyl sugar moiety (2′-MOE). However, the orphan nucleotide preferably does not comprise the 2′-OMe modification, and in yet a further preferred aspect, at least one and in a preferred aspect both the two neighbouring nucleotides flanking each nucleotide opposing the target adenosine further do not comprise a 2′-OMe modification. Complete modification wherein all nucleotides of the AON hold a 2′-OMe modification results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group, or by providing a guanine or adenine as opposing base, as these two nucleobases are also able to reduce editing of the opposing adenosine. Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. MP linkages can be formed using known chemistries, for example as disclosed in Agrawal et al. 1997. Proc Natl Acad Sci USA 94:2620-2625. In a preferred aspect, the AON of the present invention comprises 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

It is known in the art, that RNA editing entities (such as human ADAR enzymes) edit dsRNA structures with varying specificity, depending on a number of factors. One important factor is the degree of complementarity of the two strands making up the dsRNA sequence. Perfect complementarity of the two strands usually causes the catalytic domain of hADAR to deaminate adenosines in a non-discriminative manner, reacting more or less with any adenosine it encounters. The specificity of hADAR1 and 2 can be increased by introducing chemical modifications and/or ensuring a number of mismatches in the dsRNA, which presumably help to position the dsRNA binding domains in a way that has not been clearly defined yet. Additionally, the deamination reaction itself can be enhanced by providing an AON that comprises a mismatch opposite the adenosine to be edited. The mismatch is preferably created by providing a targeting portion having a cytidine opposite the adenosine to be edited. As an alternative, also uridines may be used opposite the adenosine, which, understandably, will not result in a ‘mismatch’ because U and A pair. Upon deamination of the adenosine in the target strand, the target strand will obtain an inosine which, for most biochemical processes, is “read” by the cell's biochemical machinery as a G. Hence, after A to I conversion, the mismatch has been resolved, because I is perfectly capable of base pairing with the opposite C in the targeting portion of the oligonucleotide construct according to the invention. After the mismatch has been resolved due to editing, the substrate is released and the oligonucleotide construct-editing entity complex is released from the target RNA sequence, which then becomes available for downstream biochemical processes, such as splicing and translation. Also, this on/off rate is important because the targeting oligonucleotide should not be too tightly bound to the target RNA. The desired level of specificity of editing the target RNA sequence may depend from target to target. Following the instructions in the present patent application, those of skill in the art will be capable of designing the complementary portion of the oligonucleotide according to their needs, and, with some trial and error, obtain the desired result.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof such as hADAR1 p110 and p150. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. Human ADAR3 (hADAR3) has been described in the prior art, but reportedly has no deaminase activity. It is known that hADAR1 exists in two isoforms; a long 150 kDa interferon inducible version and a shorter, 100 kDa version, that is produced through alternative splicing from a common pre-mRNA. Consequently, the level of the 150 kDa isoform present in the cell may be influenced by interferon, particularly interferon-gamma (IFN-γ). hADAR1 is also inducible by TNF-α. This provides an opportunity to develop combination therapy, whereby IFN-γ or TNF-α and AONs according to the invention are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the AONs according to the invention for the recognition domain of the editing molecule. The exact modification may be determined through some trial and error and/or through computational methods based on structural interactions between the AON and the recognition domain of the editing molecule. In addition, or alternatively, the degree of recruiting and redirecting the editing entity resident in the cell may be regulated by the dosing and the dosing regimen of the AON. This is something to be determined by the experimenter (in vitro) or the clinician, usually in phase I and/or II clinical trials.

The invention concerns the modification of target RNA sequences in eukaryotic, preferably metazoan, more preferably mammalian, most preferably human cells. The invention can be used with cells from any organ e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject. The cell can be located in vitro, ex vivo or in vivo. One advantage of the invention is that it can be used with cells in situ in a living organism, but it can also be used with cells in culture. In some embodiments, cells are treated ex vivo and are then introduced into a living organism (e.g. re-introduced into an organism from whom they were originally derived). The invention can also be used to edit target RNA sequences in cells within a so-called organoid. Organoids can be thought of as three-dimensional in vitro-derived tissues but are driven using specific conditions to generate individual, isolated tissues (e.g. see Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound be theory, the RNA editing through hADAR1 and hADAR2 is thought to take place on primary transcripts in the nucleus, during transcription or splicing, or in the cytoplasm, where e.g. mature mRNA, miRNA or ncRNA can be edited. Different isoforms of the editing enzymes are known to localize differentially, e.g. with hADAR1 p110 found mostly in the nucleus, and hADAR1 p150 in the cytoplasm. The RNA editing by cytidine deaminases is thought to take place on the mRNA level.

Many genetic diseases are caused by G to A mutations, and these are preferred target diseases because adenosine deamination at the mutated target adenosine will reverse the mutation to a codon giving rise to a functional, full length and/or wild type protein, especially when it concerns PTCs. Preferred examples of genetic diseases that can be prevented and/or treated with oligonucleotides according to the invention are any disease where the modification of one or more adenosines in a target RNA will bring about a (potentially) beneficial change. Especially preferred are Usher syndrome and CF, and more specifically the RNA editing of adenosines in the disease-inducing PTCs in CFTR RNA is preferred. Those skilled in the art of CF mutations recognise that between 1000 and 2000 mutations are known in the CFTR gene, including G542X, W1282X, R553X, R1162X, Y122X, W1089X, W846X, W401X, 621+1G>T or 1717-1G>A.

It should be clear, that targeted editing according to the invention can be applied to any adenosine (or cytosine), whether it is a mutated or a wild-type nucleotide in a given sequence.

For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether or not to prevent, delay or treat disease or for any other purpose, in medicine or biotechnology, as diagnostic, prophylactic, therapeutic, research tool or otherwise, are encompassed by the present invention.

The amount of AON to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of AON could compete for binding to a nucleic acid editing entity (e.g. ADAR) within a cell, thereby depleting the amount of the entity which is free to take part in RNA editing, but routine dosing trials will reveal any such effects for a given AON and a given target.

One suitable trial technique involves delivering the AON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively the change may be assessed on the level of the protein (length, glycosylation, function or the like), or by some functional read-out, such as a(n) (inducible) current, when the protein encoded by the target RNA sequence is an ion channel, for example.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

AONs of the invention are particularly suitable for therapeutic use, and so the invention provides a pharmaceutical composition comprising an AON of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g. syringe, inhaler, nebuliser) which includes a pharmaceutical composition of the invention.

The invention also provides an AON of the invention for use in a method for making a change in a target RNA sequence in a mammalian, preferably a human cell, as described herein. Similarly, the invention provides the use of an AON of the invention in the manufacture of a medicament for making a change in a target RNA sequence in a mammalian, preferably a human cell, as described herein.

The invention also relates to a method for the deamination of at least one specific target adenosine present in a target RNA sequence in a cell, the method comprising the steps of: providing the cell with an AON according to the invention; allowing uptake by the cell of the AON; allowing annealing of the AON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The AON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, a gel, an eye-drop, or in any other form compatible with pharmaceutical use in humans.

### EXAMPLES

### Example 1: Antisense Oligonucleotides (AONs) Comprising Methylphosphonate (MP) Linkage Modifications are More Stable than AONs Lacking Such MP Modifications, Using an In Vitro Biochemical Breakdown Assay

It is known that the presence of a 2′-OMe modification of the sugar moiety of the nucleotide opposite the target adenosine in a target RNA molecule reduces the deamination of that particular target adenosine to an inosine in comparison to an AON not carrying such 2′-OMe modification. Unfortunately, the absence of such a sugar modification at this particular position renders the AON unstable. The inventors of the present invention questioned whether such could be solved by having an internucleotide linkage modification between the two DNA nucleotides instead. For this, two methylphosphonate (MP) linkages were introduced between the two DNA nucleosides (one of which is opposite the target adenosine present in a target mouse IDUA RNA molecule) and their respective 3′ neighbouring nucleosides, in the AON. The structure of a MP-modified DNA-DNA linkage is shown in FIG. 1. FIG. 2 shows the sequences of the mouse IDUA target molecule as well as the complementary AONs used, indicating the absence of modifications between the DNA nucleosides (in capitals in FIG. 2) in AON ADAR102-1, and the presence of the MP modifications between the two DNA nucleosides and their respective 3′ neighbouring nucleosides, marked with \hat{ } in ADAR102-13. The sequence of ADAR102-21 (carrying a 2′-OMe modification in all positions of the AON as well as PS modifications between this cytidine and its 3′ neighbouring adenosine and in the two 5′ preceding linkages) and the sequence of ADAR102-25 (carrying two DNA nucleosides at the same position as ADAR102-1 and with PS linkages similar to ADAR102-21) are not shown.

All four oligonucleotides, ADAR102-1 (2×DNA, no linkage modification), ADAR102-25 (2×DNA, PS modifications), ADAR102-21 (PS modifications) and ADAR102-13 (2×DNA, MP modifications) were tested in a biochemical stability assay. All oligonucleotides were diluted with nuclease-free water (Ambion) to a concentration of 25 μM, and 10 μl of each oligonucleotide was incubated with a 10 μl of Liver lysate and a 10 μl of Nucleases Mix at 37° C. The Liver lysate was prepared as follows: 3.5 g of wild type C57Bl/6Jmouse liver was homogenized using GentleMacs homogenizer from Miltenyi Biotech in 9 ml of 100 mM Tris-HCl, pH8 and 1 mM MgOAc 4° C., centrifuged for 5 min at 4000 rpm at 4° C. and then aliquoted into 1 ml aliquots as a determined by Pierce BCA Protein Assay Kit 12 mg/ml protein concentrate and stored at −80° C. for further use. The Nucleases Mix was prepared as follows: 5 μl of a Phosphodiesterase I from Crotalus adamenteus venom (Sigma), 5 μl of a DNase I (New England BioLAbs), 5 μl of a RNase A (Thermo Scientific) and 5 μl of a Nuclease BAL-31 (New England BioLAbs) were together mixed in 30 μl of Nuclease BAL-31 buffer (New England BioLAbs) and 10 μl of nuclease-free water (Ambion). Incubation of the oligonucleotides was stopped at predetermined time points (0, 30 and 120 minutes) by adding an equal volume of denaturing sample buffer (8 M Urea, 20 mM EDTA, 5 mM Tris-HCl, pH7.5, 30% Glycerol, 0.005% Xylene cyanol, 0.01% Bromophenol Blue). Subsequently, the samples were resolved on denaturing 15% Mini-PROTEAN TBE-Urea Gel, 15 well, 15 μl, BioRad using BioRad Mini PROTEAN Tetra Cell gel electrophoresis system. The gels were stained for 30 min with Toluidine Blue O (Sigma-Aldrich) and destained in water. The gels were imaged by BioRad Gel Doc XR+ imager with White Light Conversion Screen and analysed by using the ImageJ program.

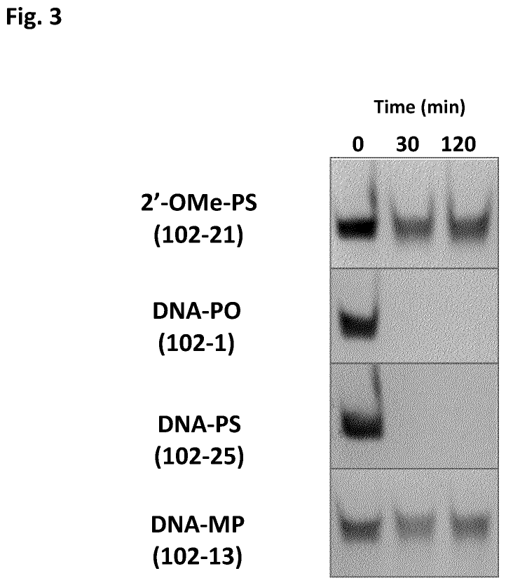

Results are given in FIG. 3, and clearly show that the presence of a PS linkage between the two DNA nucleotides (in ADAR102-25) does not give an additional stability when compared to ADAR102-1 in which the two DNA nucleotides are linked with a normal phosphodiester linkage under the conditions tested. The upper panel shows the results with ADAR102-21, carrying the 2′-OMe modifications as well as PS linkages and indicates that indeed the presence of 2′-OMe adds to the stability of the oligonucleotide. Surprisingly, the oligonucleotide carrying no 2′-OMe modification at the same positions in the oligonucleotides, but wherein MP linkages were introduced, rendered the oligonucleotides very stable, and appeared to be nearly as stable as the 2′-OMe modified oligonucleotides in this stability assay. It was concluded that the introduction of MP linkages in the oligonucleotide provided stability which was comparable to the 2′-OMe sugar modification.

### Example 2: RNA Editing by an AON Carrying Stabilizing MP Linkage Modifications

The inventors next questioned whether the MP modifications, although providing a more stable AON, would still prevent RNA editing, similar to the low RNA editing efficiencies that was observed with AONs that carry PS modifications between the DNA nucleotides in the AON (data not shown). Hence, it was investigated whether the MP modification—now it was known that it increased the stability of the AON—would allow RNA editing, in contrast to the 2′-OMe modification that (although giving stability) renders the AON ineffective in RNA editing.

For this, ADAR102-1 and ADAR102-13 were compared in an RNA editing assay as follows. First, both AONs were annealed to the mouse IDUA target RNA. Annealing was done in a buffer (5 mM Tris-Cl pH7.4, 0.5 mM EDTA and 10 mM NaCl) at the ratio 1:3 of target RNA to AON (final concentrations in the editing reaction 6 nM AON and 2 nM target). The samples were heated at 95° C. for 3 min and then slowly cooled down to RT. Next, the editing reaction was carried out. The annealed double stranded AON/target RNA was mixed with protease inhibitor (cOmplete, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH7.4, 1.5 mM EDTA, 3% glycerol, 60 mM KCl, 0.003% NP-40, 3 mM MgCl₂ and 0.5 mM DTT). The reaction was started by adding purified ADAR2, which was produced by GenScript, to a final concentration of 8 nM into the mix and incubated for predetermined time points (0, 2, 5, 10, 20, 40 and 60 min) at 37° C. The reaction was stopped by adding 190 μL boiling water and then the mixture was incubated for 5 min at 95° C. The stopped reaction mixture was then used as template for cDNA synthesis using Maxima reverse transcriptase and hexamer (Thermo Fisher). The cDNA was diluted 10× and 1 μL of this dilution was used as template for digital droplet PCR (ddPCR). The ddPCR assay for absolute quantification of nucleic acid target sequences was performed using BioRad's QX-200 Droplet Digital PCR system. 1 μl of diluted cDNA obtained from the RT cDNA synthesis reaction was used in a total mixture of 20 μl of reaction mix, including the ddPCR Supermix for Probes no dUTP (Bio Rad), a Taqman SNP genotype assay with the following forward and reverse primers combined with the following gene-specific probes:

Forward primer:
(SEQ ID NO: 4)
5′-CTCACAGTCATGGGGCTC-3′
Reverse primer:
(SEQ ID NO: 5)
5′-CACTGTATGATTGCTGTCCAAC-3′
Wild type probe (FAM NFQ labeled):
(SEQ ID NO: 6)
5′-AGAACAACTCTGGGCAGAGGTCTCA-3′
Mutant probe (HEX NFQ labeled):
(SEQ ID NO: 7)
5′-AGAACAACTCTAGGCAGAGGTCTCA-3′

A total volume of 20 μl PCR mix including cDNA was filled in the middle row of a ddPCR cartridge (BioRad) using a multichannel pipette. The replicates were divided by two cartridges. The bottom rows were filled with 70 μl of droplet generation oil for probes (BioRad). After the rubber gasket replacement, droplets were generated in the QX200 droplet generator. 40 μl of oil emulsion from the top row of the cartridge was transferred to a 96-wells PCR plate. The PCR plate was sealed with a tin foil for 4 sec at 170° C. using the PX1 plate sealer, followed by the following PCR program: 1 cycle of enzyme activation for 10 min at 95° C., 40 cycles denaturation for 30 sec at 95° C. and annealing/extension for 1 min at 63.8° C., 1 cycle of enzyme deactivation for 10 min at 98° C., followed by a storage at 8° C. After PCR the plate was read and analyzed with the QX200 droplet reader.

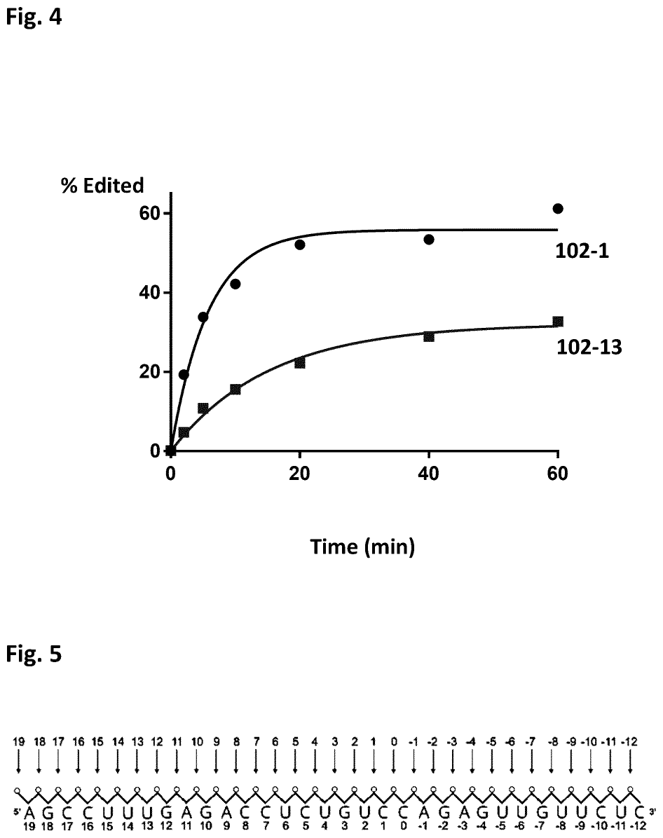

Results are given in FIG. 4 and clearly show that, albeit somewhat reduced in comparison to the AON carrying no linkage modification between the DNA nucleotides opposite the target adenosine (ADAR102-1), the use of ADAR102-13 did give RNA editing up to 30%. This clearly shows that the presence of an MP modification linking the two DNA nucleotides opposite the target adenosine in a target RNA molecule, with their respective 3′ neighbouring nucleosides, allowed RNA editing. Together with the fact that the modification increases the stability of the AON, it is held that these two features together provide an improved RNA editing tool in comparison to what had thus far been demonstrated in the prior art.

### Example 3: RNA Editing by AONs Carrying Stabilizing MP Linkage Modifications

The inventors next questioned where MP modifications could be made within the AON and still allow for RNA editing. AONs having MP modifications at linkage positions 0 to −6 relative to the orphan nucleoside were synthesised and tested. Editing assays were carried out as in Example 2, with the following changes: The final concentrations were 1 nM target RNA, 24 nM AON, and 3 nM ADAR2, and 3 mM MgSO₄ was used instead of 3 mM MgCl₂ in the editing reaction buffer. The reactions were performed as described in Example 2, and were stopped by adding 95 μl of boiling 3 mM EDTA solution into 5 μl of aliquots taken from the reactions at time points 0 s, 30 s, 1 min, 2 min, 5 min, 10 min, 25 min, and 50 min.

A 6 μl aliquot of the stopped reaction mixture was then used as template for cDNA synthesis using Maxima reverse transcriptase kit (Thermo Fisher) with a target RNA-specific primer (5′-GGAAACGTAGGTTGGGGTGTG-3′ SEQ ID NO:8). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C.

Products were amplified for pyrosequencing analysis by PCR, using the Amplitaq gold 360 DNA Polymerase kit (Applied Biosystems) according to the manufacturer's instructions, with 1 μl of the cDNA as template. The following primers were used at a concentration of 10 μM: Pyroseq Fwd2 IDUA, 5′-AGTACTCACAGTCATGGGGCTCA-3′ (SEQ ID NO:9), and Pyroseq Rev2 IDUA Biotin, 5′-GCCAGGACACCCACTGTATGAT-3′ (SEQ ID NO:10). The latter primer also contains a biotin conjugated to its 5′ end, as required for the automatic processing during the pyrosequencing reactions. The PCR was performed using the following thermal cycling protocol: Initial denaturation at 95° C. for 5 min, followed by 40 cycles of 95° C. for 30 s, 60° C. for 30 s and 72° C. for 30 s, and a final extension of 72° C. for 7 min.

As inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and the following data analysis were performed by the PyroMark Q48 Autoprep instrument (QIAGEN) following the manufacturer's instructions, with 10 μl input of the PCR product and 4 μM of the following sequencing primer: IDUA-Seq2, 5′-TGGGGCTCATGGCCCT-3′ (SEQ ID NO:11). The settings specifically defined for this target RNA strand included two sets of sequence information. The first of these defines the sequence for the instrument to analyze, in which the potential for a particular position to contain either an adenosine or a guanosine is indicated by a “/”: 5′-GTTGGATGGAGAACAAC TCTA/GGGCAGAGGTCTCAA/GAGGCTGGGGCT-3′ (SEQ ID NO:12). Note that two positions are analysed in the sequencing: The target site and a control site which should not be edited (results only shown for the target site). The second set defines the order in which the sequencing reagents corresponding to each nucleotide are to be dispensed, and also includes blank controls (i.e. nucleotides that should not be incorporated at that particular position), which is used by the instrument to define the background signal. The dispensation order was defined for this analysis as follows:

(SEQ ID NO: 13)
CGTGATGAGACACTCGTAGCAGAGTCTGCAGAGCTGCA.

The analysis performed by the instrument provides the results for the selected nucleotide as a percentage of adenosine and guanosine detected in that position, and the extent of A-to-I editing at a chosen position will therefore be measured by the percentage of guanosine in that position.

The results in FIG. 6 and FIG. 7 show that AONs that have an MP linkage between any of the nucleotides in the region in proximity of the orphan nucleotide allow RNA editing. Modifications at positions 0 and −2 were observed to provide similar RNA editing properties as the positive control AON. The other position can carry MP. They appear not as efficient in RNA editing, either in terms of the final level of editing achieved, or in the kinetics of the reaction.

### Example 4: RNA Editing in Cells by AONs Carrying Stabilizing MP Linkage Modifications

The inventors next investigated the ability of AONs to conduct editing in cells, in which the RNA with the target site sequence used in Examples 2 and 3 is present. The cells in question are a mouse embryonic fibroblast cell line, in which the endogenous Idua gene has a G-to-A mutation (creating the A at the target site) resulting in a formation of a premature stop codon (W392X). These cells additionally overexpress the Idua W392X RNA from a stably integrated cDNA construct. Briefly, 150,000 cells were seeded 24 hours before transfection, which was performed with 100 nM AON and Lipofectamine 2000 (Invitrogen) according to the manufacturer's instructions (at a ratio of 1 μl Lipofectamine 2000 to 1 μg EON). RNA was extracted from cells 48 hours after transfection using the Direct-zol RNA MiniPrep (Zymo Research) kit according to the manufacturer's instructions, and cDNA prepared using the Maxima reverse transcriptase kit (Thermo Fisher) according to the manufacturer's instructions, with a combination of random hexamer and oligo-dT primers. The cDNA was diluted 4× and 1 μL of this dilution was used as template for digital droplet PCR (ddPCR), which was performed as detailed in Example 2.

The results in FIG. 8 show that an AON with no MP (IDUA103) shows variable editing, not clearly differentiated from the background, which is defined by control samples with either non-treated (NT) cells or cells only treated with Lipofectamine 2000 (mock). In contrast, an AON bearing MP linkage modifications can facilitate editing in cells above this background level. This represents the ability of the AON to withstand nuclease degradation as well as the ability to recruit endogenous editing enzymes.

1. An antisense oligonucleotide (AON) capable of forming a double stranded complex with a target nucleic acid molecule in a cell, for use in the deamination of a target nucleotide in the target nucleic acid molecule, preferably an adenosine, wherein the nucleotide in the AON that is directly opposite the target nucleotide is the orphan nucleotide, wherein the internucleoside linkage numbering is such that linkage number 0 is the linkage 5′ from the orphan nucleotide, and wherein the linkage positions in the oligonucleotide are positively (+) incremented towards the 5′ end and negatively incremented towards the 3′ end, and wherein the AON comprises one or more methylphosphonate (MP) linkages at linkage positions 0, −1, −2, −3, −4, −5 and/or −6, wherein the MP linkage has the following chemical structure:

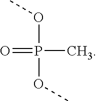

2. The AON according to claim 1, wherein the orphan nucleotide does not carry a 2′-OMe or 2′-MOE ribose modification.

3. The AON according to claim 1 or 2, wherein the AON comprises MP linkages at linkage positions −0 and/or −2.

4. The AON according to any one of claims 1 to 3, wherein the MP linkage connects a DNA nucleoside with another nucleoside.

5. The AON according to claim 4, wherein the MP linkage connects a DNA nucleoside with a DNA nucleoside.

6. The AON according to any one of claims 1 to 5, wherein the AON further comprises at least one phosphorothioate or a phosphonoacetate internucleoside linkage, and/or at least one nucleotide comprising an unlocked nucleic acid (UNA) ribose modification.

7. The AON according to any one of claims 1 to 6, wherein the AON further comprises one or more nucleotides comprising a substitution at the 2′ position of the ribose, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

8. The AON according to any one of claims 1 to 7, wherein the AON comprises at least one nucleotide comprising a 2′-OMe or a 2′-MOE ribose modification, and wherein the orphan nucleotide does not carry a 2′-OMe or a 2′-MOE ribose modification.

9. The AON according to any one of claims 1 to 8, wherein the AON is capable of, in a cell, engaging an enzyme with deaminase activity, preferably an enzyme with adenosine deaminase activity, such as human ADAR1 or ADAR2.

10. The AON according to any one of claims 1 to 9, wherein the AON is at least 10, 11, 12, 13, 14, 15, 16, 17, 18, 19, 20, 21, 22, 23, 24, 25, 26, 27, 28, 29, 30, 31, or 32 nucleotides in length, and wherein the AON is shorter than 100 nucleotides, preferably shorter than 60 nucleotides.

11. A pharmaceutical composition comprising an AON according to any one of claims 1 to 10, and a pharmaceutically acceptable carrier.

12. An AON according to any of claims 1 to 10, or a pharmaceutical composition according to claim 11, for use in the treatment or prevention of a genetic disorder, preferably selected from the group consisting of: Cystic fibrosis, Hurler Syndrome, alpha-1-antitrypsin (A1AT) deficiency, Parkinson's disease, Alzheimer's disease, albinism, Amyotrophic lateral sclerosis, Asthma, B-thalassemia, CADASIL, Charcot-Marie-Tooth disease, Chronic Obstructive Pulmonary Disease (COPD), Distal Spinal Muscular Atrophy (DSMA), Duchenne/Becker muscular dystrophy, (Dystrophic) Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber Congenital Amaurosis (such as LCA10), Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anemia, Spinal Muscular Atrophy, Stargardt disease, Tay-Sachs Disease, Usher syndrome (such as Usher syndrome type I, type II, and type Ill), X-linked immunodeficiency, Sturge-Weber Syndrome, and cancer.

13. A method for the deamination of at least one target nucleotide, preferably an adenosine, present in a target RNA molecule in a cell, the method comprising the steps of:
(i) providing the cell with an AON according to any one of claims 1 to 10, or a pharmaceutical composition according to claim 11;
(ii) allowing annealing of the AON to the target RNA molecule;
(iii) allowing a mammalian enzyme with nucleotide deaminase activity to deaminate the target nucleotide in the target RNA molecule; and
(iv) optionally identifying the presence of the deaminated nucleotide in the target RNA molecule.

14. The method of claim 13, wherein step (iv) comprises:
a) sequencing a region of the target RNA molecule, wherein the region comprises the deaminated target nucleotide;
b) assessing the presence of a functional, elongated, full length and/orwild type protein when the target nucleotide is an adenosine located in a UGA or UAG stop codon, which is edited to a UGG codon through the deamination;
c) assessing the presence of a functional, elongated, full length and/orwild type protein when two target adenosines are located in a UAA stop codon, which is edited to a UGG codon through the deamination of both target adenosines;
d) assessing, when the target RNA molecule is pre-mRNA, whether splicing of the pre-mRNA was altered by the deamination; or
e) using a functional read-out, wherein the target RNA molecule after the deamination encodes a functional, full length, elongated and/or wild type protein.

15. A method for the deamination of at least one target nucleotide, preferably an adenosine, present in a target RNA molecule, the method comprising the steps of:
(i) providing an AON according to any one of claims 1 to 10;
(ii) allowing annealing of the AON to the target RNA molecule;
(iii) allowing a mammalian enzyme with nucleotide deaminase activity to deaminate the target nucleotide in the target RNA molecule; and
(iv) identifying the presence of the deaminated nucleotide in the target RNA molecule.
